(19) United States
(12) Patent Application Publication
(10) Pub. No.: US 2009/0054259 A1
(43) Pub. Date: Feb. 26, 2009

(54) METHODS FOR INDUCING COMPLETE APOPTOSIS OF LIVER CELLS EXPRESSING HBCHI PROTEIN AND SCREENING FOR SUBSTANCES OR GENES THAT INHIBIT THE APOPTOSIS

(76) Inventors: Yeon-Soo Kim, Seoul (KR); Sukyung Lee, Seoul (KR); Jung Me Hwang, Daejeon (KR); Young-Kwan Lee, Daejeon (KR); Hyeseong Cho, Kyungki-do (KR); Ki-Sun Kwon, Daejeon (KR)

Correspondence Address: SUGHRUE MION, PLLC, 2100 PENNSYLVANIA AVENUE, N.W., SUITE 800, WASHINGTON, DC, 20037, US

(21) Appl. No.: 10/595,513

(22) PCT Filed: Apr. 9, 2004
(86) PCT No.: PCT/KR04/00830

§ 371 Date: Apr. 25, 2006

(51) Int. Cl. C40B 30/06; C12N 5/00; C12Q 1/02; C12Q 1/68

(52) U.S. Cl. 506/10; 435/375; 435/29; 435/6

(57) ABSTRACT

A complete apoptosis of liver cells expressing HBx protein is achieved with a NF-κB inhibitor treatment. This method is useful in screening for substances or genes that inhibit complete apoptosis of liver cells expressing HBx protein, and, further, the substance or gene may be employed as an active ingredient of a therapeutic composition for treating hepatitis or hepatocarcinoma induced by HBV infection.

### FIELD OF THE INVENTION

The present invention relates to a method for inducing complete apoptosis of liver cells expressing HBx protein by treating the cells with an NF-κB inhibitor.

### BACKGROUND OF THE INVENTION

Human hepatitis B virus (HBV) is one of causative agents of acute chronic hepatitis, cirrhosis, and hepatocarcinoma HBV genome protein is comprised of four proteins: HBs, HBc, HBx, and polymerase. Among these proteins, HBx protein, in particularly, has arrested considerable attention for its pleiotypic (both-sided) function in controlling cell death (apoptosis) and cell growth (proliferation) (Su and Schneider, Proc. Natl. Acad. Sci. USA, 94, 8744-8749, 1997). There are some evidences that hepatocytes infected by HBV undergo apoptosis, and some of the hepatocytes escape from apoptosis pathway to develop carcinoma (Chisari, Curr. Topics. Microbiol Immunol., 206, 149-173, 1996). Further, it has been reported that HBx protein promotes tumorgenesis in a transgenic mouse (Kim et. al., Nature, 353, 317-320).

It has been known that an inflammatory response by TNF-α (tumor necrosis factor α) facilitates apoptosis induced by HBx protein (Su and Schneider, Proc. Natl. Acad. Sci. USA, 94, 8744-8749, 1997; and Chisari, Curr. Topics. Microbiol. Immunol., 206, 149-173, 1996). HBx protein sensitizes cells to apoptosis by means of increasing directly or indirectly the amount of TNF-α, or regulating intermediates involved in the signaling pathway of apoptosis (Su et al., J. of Virol. 75, 215-225, 2001).

TNF-α is an inflammatory cytokine that controls various signaling pathways related to apoptosis, cell proliferation, cell differentiation, and cell survival.

TNFR (Tumor necrosis factor receptor) family members can be classified into two subfamilies based on the presence or absence of a cytoplasmic death domain therein. When TNF-α binds to TNFR-I containing the death domain, apoptosis pathway is triggered through caspase activation. While, TNFR-II lacking death domain facilitates cell survival pathway through NF-κB activation (Chandel et al., J. of Biol. Chem., 276, 42728-42736, 2001; Roth et al., Cell, 83, 1243-1252).

NP-κB is generally sequestered in the cytoplasm in an inactive complex by combining with it's inhibitory protein, IκB. Upon stimulation by a cytokine such as TNF-α, IκB gets phosphorylated and degraded. This pathway is followed by releasing and activation of NF-κB. The activated NF-κB is translocated to the nucleus where it binds to the promoter/enhancer regions of target genes, to activate transcription of the genes (Su et al., J. of Virol., 75, 215-225, 2001; Wahl et al., J. Clin. Invest., 101(5), 1163-1174, 1998).

Sulfasalazine, which is a potent and specific inhibitor of NF-κB, prevents TNF-α-induced nuclear translocation of NF-κB through inhibiting the phosphorylation of IκB (Wahl et al., J. Clin. Invest., 101(5), 1163-1174, 1998). HBx protein either directly or indirectly controls apoptosis by facilitating or inhibiting the NF-κB activation in the TNF-α mediated signal pathway.

Accordingly, the present inventors have endeavored to find out how complete apoptosis of liver cells expressing HBx protein can be achieved by blocking the cell survival pathway through the inhibition of the activation of NF-κB.

### SUMMARY OF THE INVENTION

Accordingly, it is an object of the present invention to provide a method for inducing a complete apoptosis of liver cells expressing HBx protein, by treating the cells with an NF-κB inhibitor.

It is another object of the present invention to provide a method of screening for substances or genes that inhibit apoptosis of liver cells expressing HBx protein.

### BRIEF DESCRIPTION OF THE DRAWINGS

The above and other objects and features of the present invention will become apparent from the following description of the invention, when taken in conjunction with the accompanying drawings, which respectively show:

FIG. 1: western blotting results performed with human liver cell lines Chang V9 and Chang X31 to examine the expression of HBx protein;

FIG. 2a: survival rates of Chang V9 and Chang X31 cells 120 hours after treatment with 1.0, 1.3, 1.5, 1.7 and 2.0 mM of sulfasalazine;

FIG. 2b: complete apoptosis of Chang V9 and Chang X31 cells 120 hours after being treated with 1.3 mM sulfasalazine;

FIG. 3: appearance of Chang V9 and Chang X31 cells stained with DAPI solution 72 hours after 1.3 mM sulfasalazine treatment;

FIG. 4: Chang X31 cell colonies stained with crystal violet 120 hours after 1.3 mM sulfasalazine treatment, wherein Chang X31 cells were treated with no substance, staurosporin, Glutathione and N-acetyl-L-cysteine before treatment of the sulfasalazine, respectively;

FIG. 5: transfection efficiency of lacZ gene into GP293 and the retroviral titer against Chang X31;

FIG. 6: results of Chang X31 cell colonies stained with crystal violet 120 hours after 1.3 mM sulfasalazin treatment, wherein pMYK-eGFP, GPx, PrxII and Prx III genes were introduced into Chang X31 before the treatment with sulfasalazine, respectively; and

FIG. 7: western blotting performed with Chang X 31 cell transfected with peroxyredox II (PrxII).

### DETAILED DESCRIPTION OF THE INVENTION

The present invention relates to a method for inducing a complete apoptosis of liver cells expressing HBx protein, which comprises treating the cells with an NF-κB inhibitor.

The present invention also relates to a method of screening for substances that inhibit apoptosis of liver cells expressing HBx protein, which comprises treating the cells with a candidate substance before or after the NF-κB inhibitor treatment.

The present invention further relates to a method of screening for genes that inhibit apoptosis of liver cells expressing HBx protein, which comprises introducing a candidate gene into the liver cells, and treating the liver cells with an NF-κB inhibitor.

As used, the term “complete apoptosis” refers to 100% apoptosis, unless otherwise indicated.

In the present invention, a cell line expressing HBx protein is prepared by introducing a vector comprising HBx gene into a Chang human liver cell line. In order to examine whether HBx protein is stably expressed in the liver cells, western blotting is performed with cell extracts of each cloned cell. The liver cell line stably expressing HBx protein, is designated as Chang X31; and the cell line comprising a control vector which does not contain HBx gene, is identified as Chang V9.

It has been known that HBx protein induces TNF-α mediated apoptosis by facilitating TNF-α gene expression (Su and Schneider, Proc. Natl. Acad. Sci. USA, 94, 8744-8749, 1997).

TNF-α, on the other hand, facilitates cell survival by activating the transcriptional factor NF-κB. Accordingly, TNF-α plays the dual role of mediating two conflicting signal transduction pathways, i.e., cell survival and apoptosis. Even when treated with a high concentration of TNF-α, complete apoptosis does not occur, suggesting that TNF-α mediates the cell survival pathway as well as the cell death pathway. Accordingly, TNF-α is not capable of inducing a complete apoptosis. Thus, in order to induce a complete apoptosis of liver cells expressing HBx protein, the cells must be treated with a substance which blocks the signal transduction for cell survival.

In respect to the above need, the present invention provides a method for inducing a complete apoptosis of liver cells expressing HBx protein by the treatment with an NF-κB inhibitor which blocks the signal pathway involved in cell survival.

Exemplary NF-κB inhibitors that may be employed in present invention include sulfasalazine (Wabl et al., J. Clin. Invest., 101(5), 1163-1174, 1998; and Cavallini et al., Biochem Pharmacol, 62(1), 141-147, 2001), sanguinarine (Chaturvedi et al., J. Biol. Chem., 272(48), 30129-30134, 1997), oleandrin (Manna et al., Cancer Res., 60(14), 3838-3847, 2000), salicylate (Yin et al., Nature, 396(6706), 77-80, 1998; Cavallini et al., Biochem. Pharmacol., 62(1), 141-147, 2001; and Kopp et al., Science, 265(5174), 956-959, 1994), mesalazine (Bantel et al., Am. J. Gastroenterol., 95(12), 3343-3345), aspirine (Yin et al., Nature, 396(6706), 77-80, 1998; and Kopp et al., Science, 265(5174), 956-959, 1994) and methotrexate (Majumdar, J. Immunol., 167(5), 2911-2920, 2001). The preferred NF-κB inhibitor is sulfasalazine.

In the present invention, complete apoptosis of the cells expressing HBx protein is achieved by a NF-κR inhibitor treatment.

The present invention provides a condition for inducing complete apoptosis within 120 hours after the sulfasalazine treatment, and, in particular, an optimum concentration of sulfasalazine for inducing complete apoptosis of Chang X31 cells, while the morphological features accompanying apoptosis (e.g., nucleus condensation, shrinkage and fragmentation) are defined.

The present invention also relates to a method of screening for substances that inhibit the apoptosis of the liver cells expressing HBx protein, by employing the above method for inducing complete apoptosis, which comprises the steps of: a) treating the liver cells expressing HBx protein with a candidate substance before or after the treatnent with an NF-κB inhibitor; and b) examining whether the apoptosis is protected. Preferably, the NF-κB inhibitor is sulfasalazine.

If the candidate substance inhibits apoptosis under the condition for inducing complete apoptosis, the substance may be employed as an active ingredient of a therapeutic composition for treating hepatitis and hepatocarcinoma caused by HBV infection.

Accordingly, the above method may be employed in screening for a substance for treating hepatitis or hepatocarcinoma induced by HBV infection.

The present invention further relates to a method of screening for genes which inhibit apoptosis of the liver cells expressing HBx protein, by employing the above method for inducing complete apoptosis, which comprises the steps of: a) introducing a candidate gene into liver cells expressing HBx protein; b) treating the liver cells with an NF-RB inhibitor; and c) examining whether the apoptosis is protected. In step a), a retroviral cDNA library or an expression vector may be employed to introduce the candidate gene into the cells. Preferably, the NF-κB inhibitor is sulfasalazine. In the case of employing an expression vector, a conventional method (e.g. the liposome or calcium phosphate method) may be used.

The following Examples are intended to further illustrate the present invention without limiting its scope.

### EXAMPLE 1

### Preparation of Human Cell Line Chang X31 Expressing HBx Protein

Vectors pTetX (Kim et al., J. Bio. Chem., 273, 381-385, 1998) and pTRE (Clonetech., U.S.A) were prepared. pTetX comprises HA-tagging HBx gene, while pTRE does not comprise it.

Chang cells (ATCC CCL-13, U.S.A) were transfected with pTetX and pTRE, respectively, using the profection mammalian transfection system (Promega, U.S.A.). 24 hours after the transfection, the cells were subcultured in the 400 μl/ml of G418 (Invitrogen, U.S.A) containing medium and maintained for 3 weeks to establish the liver cells comprising HBx gene. The cell line expressing HBx protein and the cell line not expressing HBx protein were designated as Chang X31 and Chang V9, respectively.

The expression of HBx protein was confirmed by western blotting using the cells harvested 48 hours after inoculating Chang X31 and Chang V9 as samples.

In western blotting, HA antibody and anti-rabbit second antibody were employed as a first monoclonal antibody and a secondary antibody, respectively. The samples were reacted for 1 day with HA antibody diluted in 5% of skim milk solution by the ratio of 1:1,000, and then for 1 hour with the anti-rabbit secondary antibody diluted in 5% of skim milk solution by the ratio of 1:1,000. Then, the samples were subjected to a luminescent reaction using ECL Kit (Enhanced Chemiluminescent Kit, Amersham, U.S.A.) and exposed to X-ray films (Kodak, Germany) to detect the expression of HBx protein.

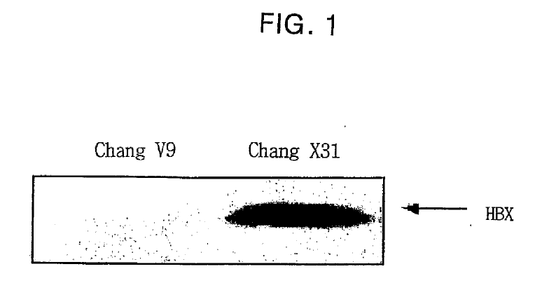

As a result, the band corresponding to HBx protein was observed only for Chang X31, not for Chang V9 (see FIG. 1).

### EXAMPLE 2

### Determination of the Condition for Inducing Complete Apoptosis of the Human Liver Cell Line Expressing HBx Protein

The condition for inducing complete apoptosis of the liver cells comprising HBx gene was determined by treating the cells with various amounts of sulfasalazine.

1×10⁵ cells of Chang X31 and 0.8×10⁵ cells of Chang V9 were inoculated on 6-well plates, respectively. 48 hours after inoculation (i.e., at the time the cell density reached about 60%), the cell cultures were treated with 1.0, 1.3, 1.5, 1.7 and 2.0 mM of sulfasalazine, respectively.

48 hours after the sulfasalazine treatment, the cells were transferred to new plates having fresh media, and then, treated again with the same concentration of sulfasalazine. 72 hours after the sulfasalazine re-treatment, the extents of the apoptosis were determined as function of the concentration of sulfasalazine: When the concentration of sulfasalazine was 1.0 mM, both Chang V9 and Chang X31 survived at rates of at least 50%; when 1.3 mM, apoptosis occurred to the extents of 10% for Chang V9 and 100% for Chang X31; and when at least 1.5 mM, apoptosis occurred to the extents of 100% for Chang X31 and at least 20% for Chang V9.

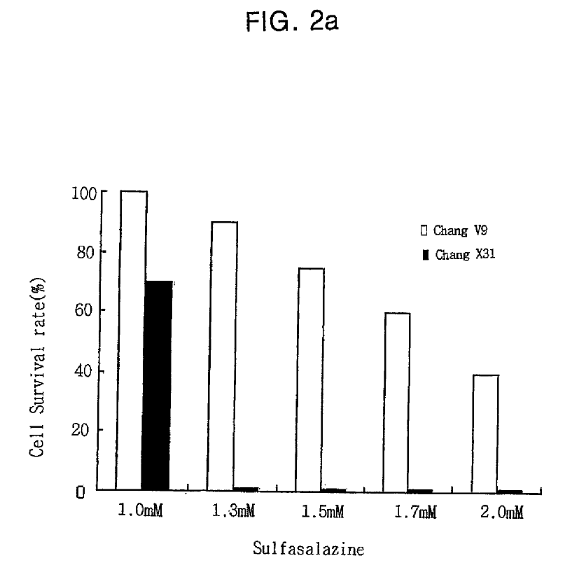

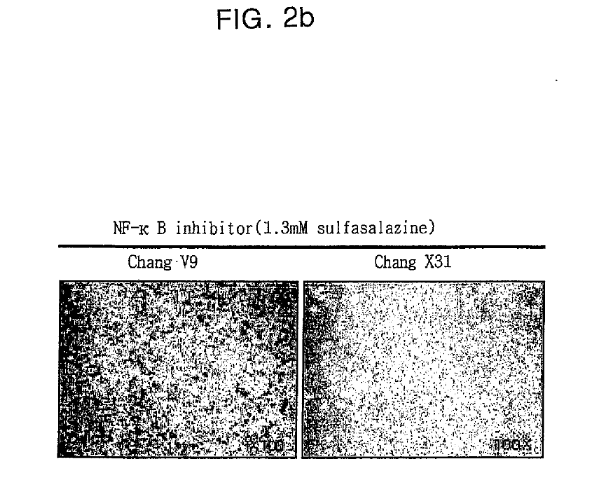

Accordingly, complete apoptosis of Chang X31 cells was induced within 120 hours, when the cells were treated with at least 1.3 mM of sulfasalazine (see FIG. 2b).

### EXAMPLE 3

### Morphology of Chang X31 Cells in which Apoptosis was Induced

Chang X 31 and Chang V9 cells were split on 6-well plates, and 48 later, treated with 1.3 mM sulfasalazine.

72 hours after the sulfasalzine treatment, DAPI staining was performed to observe apoptosis. The cells were directly fixed in the cold solution consisting of 1% formamide and 0.2% glutaraldehyde for 5 min, washed twice with PBS and then stained with 1 mg/ml of DAPI solution (Sigma, USA). The cells were incubated for 5 hours in dark.

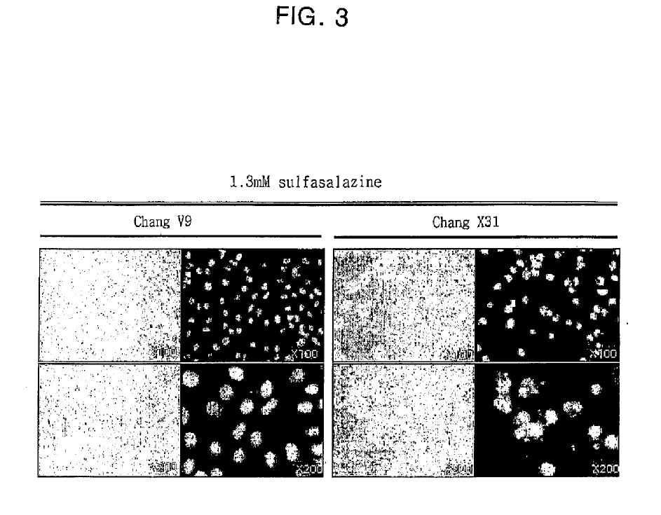

The stained cells were examined using a fluorescent microscopy. As a result the typical features of apoptosis, i.e. nuclear condensation, shrinkage, and fragmentation were observed in only Chang X 31, but not in Chang V9 (see FIG. 3).

### EXAMPLE 4

### Screening of Substances that Inhibit the Apoptosis of the Liver Cells Expressing HBx Protein

Chang X31 were prepared on 6-well plates, and 2 days later, treated with 2 mM GSH (glutathione, Sigma, U.S.A) (group I), 1 mM NAC (N-acetyl-L-cystein, Sigma, U.S.A) (group II), 0.5 mM STS (staurosporine, Sigma, U.S.A) (group III), and no substance (control group), respectively.

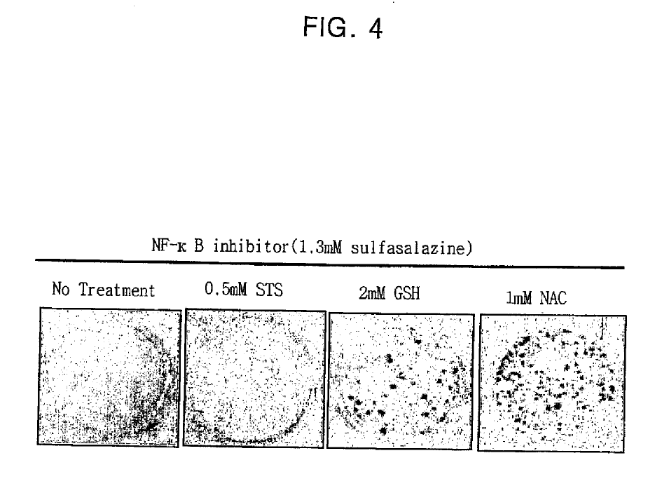

The cells of groups I, II, III and the control group were each treated with 0.5% crystal violet (Showa Kagaku, Tokyo, Japan) to observe the extent of colony formation (see FIG. 4). As a result, 100% apoptosis was observed for the control group and group III, while only partial apoptosis, for groups I and II.

Thus, it was found that the anti-oxidant, GSH or NAC, is effective in inhibiting the apoptosis of the liver cell line, while the inhibitor of protein synthesis, STS, does not inhibit the apoptosis of the liver cell line.

### EXAMPLE 5

### Screening for a Gene that Inhibits the Apoptosis of the Liver Cells Expressing HBx Protein

(1) Availability of Retroviral cDNA Library

In order to examine whether a retroviral cDNA library vector can be used for screening for a gene that inhibits the apoptosis of Chang X31 cell line, the titer of report retroviral vector MFZ/lacZ puro (Oh et al., Mol. Cells, 11(2), 192-197, 201, obtained from Dr. Jung Hee-Yong in Microbiology class of Hanyang University) was examined as follows.

Retroviral packaging cell line GP293(Clontech, USA) was transfected with 0.5 μg of MFZ/lacZ puro and 0.5 μg of pHCMV-G (the expression vector of the envelope glycoprotein) (Aiken C., J. Viol., 71(8), 5871-5877, 1997) using Lipofectamine Plus (Invitrogen, U.S.A.).

48 hours after the transfection, supernatants of cell culture comprising recombinant retrovirus expressing LacZ were collected. Chang X31 were infected with the supernatants, incubated in the presence of 8 μg/ml of polybrene for 48 hours at 37° C., directly fixed in a cold solution (1% formamide and 0.2% glutaraldehyde) for 5 min, washed twice with PBS, and then stained for 12 hours with the dye solution (4 mM of potassium ferrocyanide, 2 mM of MgCl₂ and 0.625 mg/ml of X-gal (Promega, U.S.A.)).

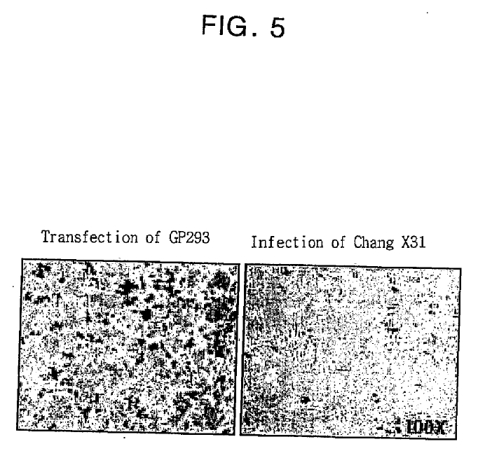

The titer of retroviral cDNA library against Chang X31 cell line was measured by counting the cells infected with total lacZ gene to be at least 1×10⁴ cfu/ml (see FIG. 5).

Thus, it was demonstrated that a retroviral cDNA library vector is available for screening for a gene that inhibits the apoptosis of Chang X31 cell line.

### (2) Screening for a Gene by the Expression Vector (Observation of the Apoptosis of the Liver Cell Line by Introducing an Antioxidant Gene)

An antioxidant gene was introduced into a mammalian expression vector to prepare a vector expressing the anti-oxidant.

Specifically, genes coding anti-oxidants, Glutathione peroxidase (GPx), PrxII (peroxyredoxin II) and PrxIII (peroxyredoxin III) were sub-cloned into mammalian expression vector pCMV/myc/cyto to prepare expression vectors pCMV/myc/cyto-GPx (the vector expressing GPx), pCMV/myc/cyto-PrxII (the vector expressing PrxII), and pCMV/myc/cyto-PrxIII (the vector expressing PrxIII), respectively (Kang et al., J. Biol. Chem. 273, 6297-6302, 1998).

1×10⁵ cells of Chang X31 cells were transfected with pCMV/myc/cyto-GPx, pCMV/myc/cyto-PrxII, pCMV/myc/cyto-PrxIII and pMYK-eGFP (a control vector containing no antioxidant gene), respectively. 36 hours later, 1.3 mM sulfasalazine was added thereto, the cells were incubated for 120 hours, and then, the extents of apoptosis were examined.

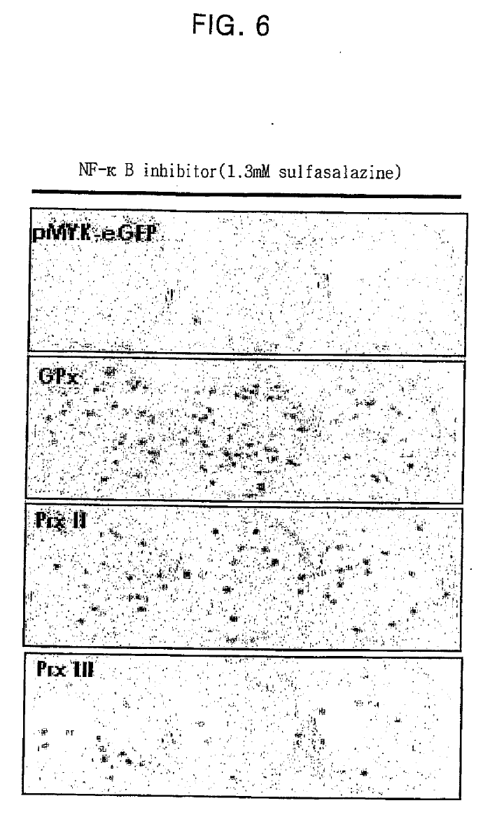

In the cells transfected with pMYK-eGFP, complete apoptosis of was observed, while in those transfected with antioxidant genes (pCMV/myc/cyto-GPx, pCMV/myc/cyto-PrxII, pCMV/myc/cyto-PrxIII), apoptosis was partially inhibited and cell colonies formed. Then, the colonies were treated with 0.5% crystal violet (Showa Kagaku, Tokyo, Japan) to examine the extent of the colony formation (see FIG. 6).

Colony formation was observed for the cells into which the genes coding the antioxidants were introduced, but not for the control group. This indicates that apoptosis of Chang X31 is inhibited by anti-oxidants (GPx, PrxII and PrxIII). Accordingly, genes coding the antioxidants may be employed to prevent or treat hepatitis or liver cancer.

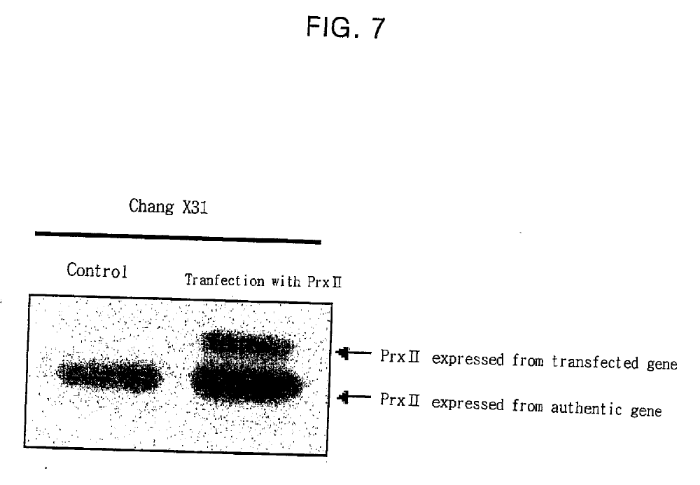

Further, it was confirmed that PrxII protein was expressed at a high level, as was observed by the western blotting analysis of the cells comprising pCMV/myc/cyto-PrxII (see FIG. 7).

While the invention has been described with respect to the above specific embodiments, it should be recognized that various modifications and changes may be made to the invention by those skilled in the art which also fall within the scope of the invention as defined by the appended claims.

What is claimed is:

1. A method for inducing complete apoptosis of a liver cell expressing HBx protein, which comprises treating the cell with an NF-κB inhibitor.

2. The method of claim 1, wherein the liver cell is Chang X31 cell line.

3. The method of claim 1, wherein the NF-κB inhibitor is sulfasalazine.

4. The method of claim 3, wherein sulfasalazine is employed in an amount of at least 1.3 mM to induce complete apoptosis within 120 hours after the treatment.

5. The method of claim 4, wherein the amount of sulfasalazine employed ranges from 1.3 to 2.0 mM.

6. A method of screening for substances that inhibit apoptosis of a liver cell expressing HBx protein, comprising the steps of:
a) treating the liver cell expressing HBx protein with a candidate substance before or after the treatment of an NF-κB inhibitor; and
b) examining whether the apoptosis is protected.

7. A method of screening for genes that inhibit apoptosis of a liver cell expressing HBx protein, comprising the steps of:
a) introducing a candidate gene into the liver cell expressing HBx protein;
b) treating the liver cell with an NF-κB inhibitor; and
c) examining whether the apoptosis is protected.

8. The method of claim 7, wherein a retroviral cDNA library or an expression vector is employed in the step of introducing the candidate gene into the cell.

9. The method of claim 6 or 7, wherein the NF-κB inhibitor is sulfasalazine.
